(19) United States
(12) Patent Application Publication
(10) Pub. No.: US 2017/0246651 A1
(43) Pub. Date: Aug. 31, 2017

(54) Fragrance Sampler

(71) Applicant: COLGATE-PALMOLIVE COMPANY, New York, NY (US)

(72) Inventor: John C. Crawford, Mahopac, NY (US)

(73) Assignee: COLGATE-PALMOLIVE COMPANY, New York, NY (US)

(21) Appl. No.: 15/511,321

(22) PCT Filed: Sep. 16, 2014
(86) PCT No.: PCT/US2014/055858

§ 371 Date: Mar. 15, 2017

(51) Int. Cl. B05B 17/06; B05B 9/00; A61L 9/03; A61L 9/14; A61L 9/12

(52) U.S. Cl. CPC B05B 17/0607; A61L 9/14; A61L 9/125; A61L 9/035; B05B 9/002; A61L 2209/132

(57) ABSTRACT

A system and method for producing a fragrance. The system may include body having a plurality of chambers defined therein. Each chamber may include a fragrance accord. A circuit and a plurality of emitters may be positioned within the body. The emitters may be connected to and controlled by the circuit, and each of the emitters may be positioned adjacent to a corresponding one of the chambers and control ejection of the fragrance accord therein.

### BACKGROUND

Fragrance tends to play a significant part in a customer's selection of a product such as shampoo or deodorant. However, oftentimes these products are packaged in such a way as to prevent the customer from smelling the fragrance of the product in the store. Rather, the customer is expected to purchase the product first before opening the packaging and smelling the fragrance. This may lead to customers that are not pleased with the fragrance of the product they have purchased. In addition, this may cause some rogue customers to tear open the packaging of the product in the store to smell the fragrance prior to purchasing the product. If the customer does not like the fragrance enough to purchase the product, the product is put back on the shelf with torn packaging, making subsequent customers less likely to purchase that particular product. What is needed is an improved system and method to enable customers to smell the fragrances of various products prior to purchasing them.

### BRIEF SUMMARY

A system for producing a fragrance is disclosed. The system may include a body having a plurality of chambers defined therein. Each chamber may include a fragrance accord. A circuit and a plurality of emitters may be positioned within the body. The emitters may be connected to and controlled by the circuit. Each of the emitters may be positioned adjacent to a corresponding one of the chambers and control ejection of the fragrance accord therein.

The emitters may be or include piezoelectric drivers, heating elements, aerosol devices, or a combination thereof. The circuit may be configured to receive a plurality of fragrance selections, each corresponding to a different fragrance. Each fragrance may include a different combination of two or more of the fragrance accords. In response to receiving a fragrance selection, the circuit may transmit a first signal to a first of the emitters, causing the first of the emitters to displace. In response to the first of the emitters displacing, a portion of a first of the fragrance accords may be dispensed from a first of the chambers adjacent to the first of the emitters. In response to receiving the fragrance selection, the circuit may also transmit a second signal to a second of the emitters, causing the second of the emitters to displace. In response to the second of the emitters displacing, a portion of a second of the fragrance accords may be dispensed from a second of the chambers adjacent to the second of the emitters. The selected fragrance may include the first fragrance accord and the second fragrance accord. The fragrance accords may be in liquid form, gel form, or solid state form.

In another embodiment, the system may include a body having a plurality of chambers defined therein. Each chamber may include a fragrance accord. A circuit may be positioned within the body, and the circuit may be configured to receive a signal corresponding to a fragrance selection from a portable electronic device. A plurality of piezoelectric drivers may also be positioned within the body. The piezoelectric drivers may be connected to and controlled by the circuit, and each of the piezoelectric drivers may be positioned adjacent to a corresponding one of the chambers.

The circuit may be configured to receive the signal when the portable electronic device is physically connected to the body. In another embodiment, the circuit may be configured to receive the signal wirelessly from the portable electronic device. The signal may be an audio signal. For example, the signal may include at least a portion of a song.

A method for producing a fragrance is also disclosed. The method may include receiving a selection of a fragrance. In response to the selection, a first signal may be transmitted to a first piezoelectric device that causes the first piezoelectric device to displace. In response to the first piezoelectric device displacing, a first fragrance accord may be dispensed from a first chamber. Also in response to the selection, a second signal may be transmitted to a second piezoelectric device that causes the second piezoelectric device to displace. In response to the second piezoelectric device displacing, a second fragrance accord may be dispensed from a second chamber. The fragrance may include the first fragrance accord and the second fragrance accord.

The selection may be received from a portable electronic device. The selection may be received wirelessly from the portable electronic device. The portable electronic device may be or include a cellular phone, a tablet, an MP3 player, or a combination thereof. The first fragrance accord and the second fragrance accord may be combined in a common chamber to form the fragrance.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 depicts a perspective view of an illustrative system for producing one or more fragrances, according to an embodiment.

FIG. 2 depicts a schematic view of the system shown in FIG. 1, according to an embodiment.

FIG. 3 depicts a partial perspective view of another illustrative system for producing one or more fragrances, according to an embodiment.

FIG. 4 depicts a schematic view of the system shown in FIG. 3, according to an embodiment.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

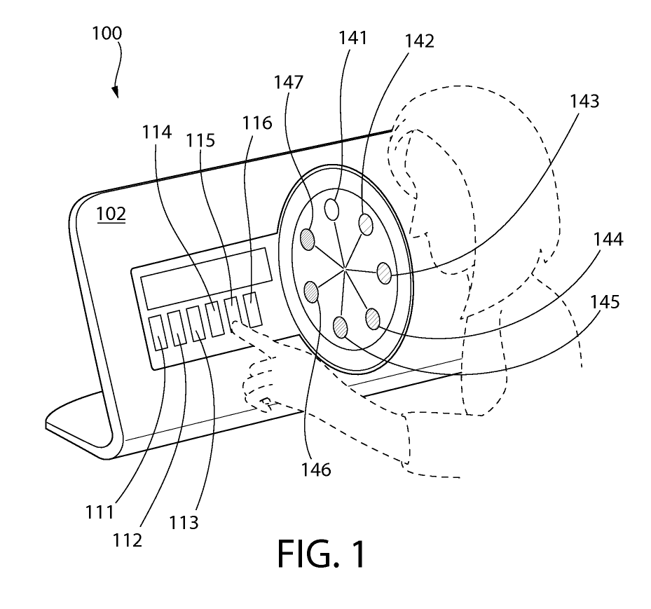

FIG. 1 depicts a perspective view of an illustrative system 100 for producing one or more fragrances, according to an embodiment. In at least one embodiment, the system 100 may be located in a store to allow customers to sample the fragrances of various products to help the customers select which product to purchase. Illustrative products may be or include soap, shampoo, conditioner, deodorant, perfume, cologne, air fresheners, household cleaning products, and the like. However, in other embodiments, the system 100 may be used to produce various fragrances and introduce them into the environment at a user's home, office, or at any other location.

The system 100 may include a body 102. The body 102 may include one or more inputs 111-116. Although six inputs 111-116 are shown, it will be appreciated that more or fewer inputs may be present. The inputs 111-116 may be buttons that may be touched or pressed. Each input 111-116 may correspond to a particular fragrance (e.g., for a particular product). For example, the input 111 may correspond to the fragrance of cucumber citrus scented dishwashing liquid manufactured and sold by the Colgate-Palmolive Company.

The body 102 may also include one or more chambers 141-147. Although seven chambers 141-147 are shown, it will be appreciated that more or fewer chambers may be present. Each chamber 141-147 may include a fragrance building block or accord 151-157 therein. The fragrance accords 151-157 may be in liquid form, gel form, or solid state form. Each fragrance may be made up of a unique combination of the fragrance accords 151-157. For example, the cucumber citrus fragrance may be made up of 40% of the first fragrance accord 151, 40% of the second fragrance accord 152, and 20% of the third fragrance accord 153.

The fragrance accords 151-157 may include amber, ambreine, aquatic, aromatic, balsam, chypre, citrus, cologne, floral, fresh, fougere, fruit, gourmand, green, herbal, mellis, oriental, spice, wood, a combination thereof, or the like. For example, the fragrance accord 151 in chamber 141 may be fruit, and the fragrance accord 152 in chamber 142 may be green.

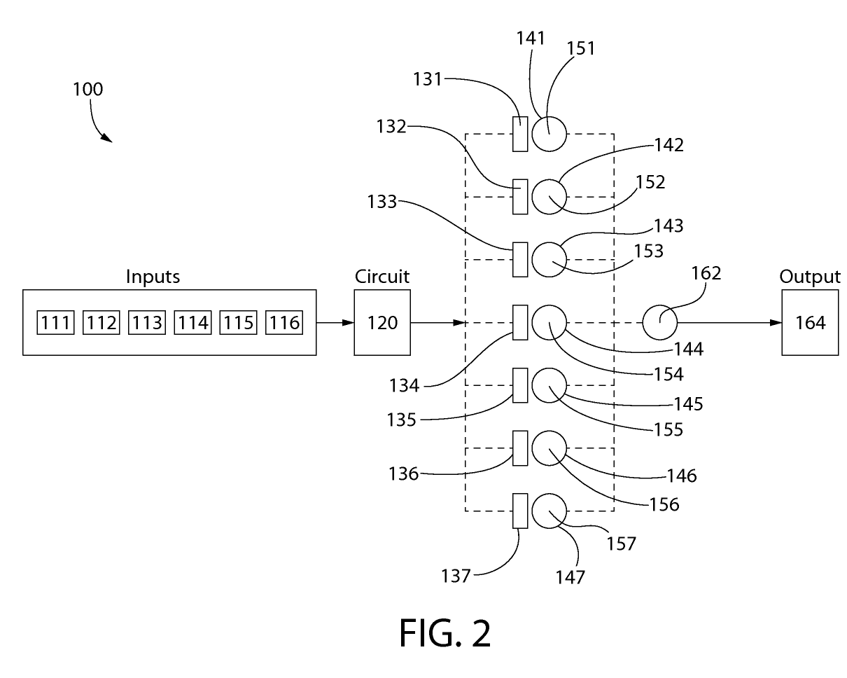

FIG. 2 depicts a schematic view of the system 100 shown in FIG. 1, according to an embodiment. Although not shown in FIG. 1, the body 102 may also include one or more circuits (one is shown: 120) and one or more actuators or emitters (seven are shown: 131-137). As shown, each of the inputs 111-116 may be connected to a common circuit 120. In another embodiment, each of the inputs 111-116 may be connected to a separate circuit. The circuit 120 may include a battery, resistors, capacitors, inductors, transistors or other switches, logic gates, buttons, LED lights, sound producing elements, a combination thereof, or the like to create a varied user experience.

The circuit 120 may be connected to the emitters 131-137. Each of the emitters 131-137 may be positioned adjacent to and/or connected to a corresponding one of the chambers 141-147. The emitters 131-137 may be or include piezoelectric drivers, heating elements, aerosol devices, and the like.

Referring to FIGS. 1 and 2, in operation, the user may select one of the inputs (e.g., input 111 corresponding to the cucumber citrus fragrance). In response to the selected input 111, the circuit 120 may transmit one or more signals to one or more of the emitters 131-137. For example, the cucumber citrus fragrance may be made up from fragrance accords 151-153 but not fragrance accords 154-157. In this instance, the circuit 120 may transmit signals to the emitters 131-133 causing the emitters 131-133 to activate (e.g., bend, vibrate, or otherwise displace). When the emitters 131-133 activate, they may cause the corresponding chambers 141-143 to dispense a portion of the fragrance accords 151-153 contained therein.

In at least one embodiment, the cucumber citrus fragrance may be made up of equal portions of the fragrance accords 151-153. In this embodiment, the signals transmitted to the emitters 131-133 may cause the emitters 131-133 to vibrate or displace with the same or similar amplitude, frequency, and/or duration. In another embodiment, the cucumber citrus fragrance may be made up of different portions of the fragrance accords 151-153. For example, the cucumber citrus fragrance may be made up of 40% of fragrance accord 151, 40% of fragrance accord 152, and 20% of fragrance accord 153. In this embodiment, the signals transmitted to the emitters 131, 132 may cause the emitters 131, 132 to vibrate or displace with twice the amplitude, frequency, and/or duration as the emitter 133 so as to output twice as much liquid as is output by the emitter 133.

The fragrance accords 151-153 dispensed from the chambers 141-143 may flow into a common chamber 162. The common chamber 162 may be a tubular passageway between the chambers 141-147 and an output 164. In at least one embodiment, the fragrance accords 151-153 may be stirred or mixed in the common chamber 162 prior to being dispensed through the output 164. The output 164 may be or include a nozzle configured to spray the fragrance into the air for the user to smell. Although not shown, in at least one embodiment, a fan may be positioned proximate to the output 164 to propel the fragrance into the air.

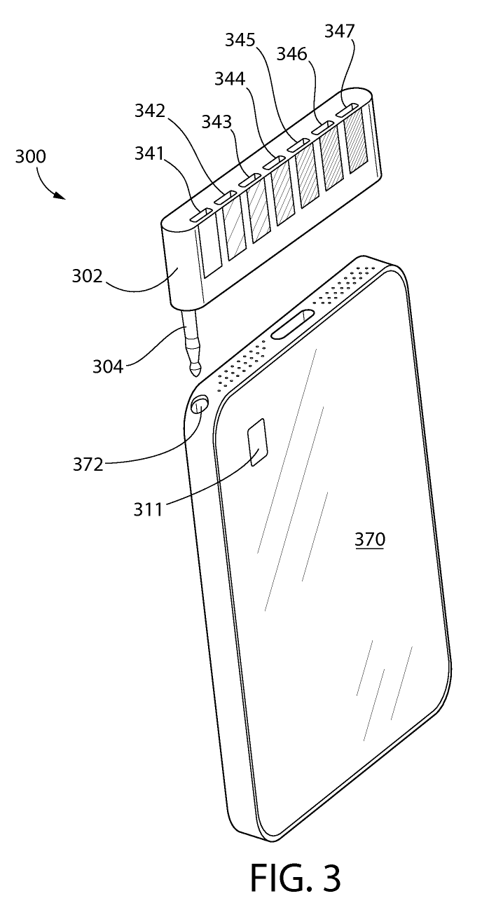

FIG. 3 depicts a partial perspective view of another illustrative system 300 for producing one or more fragrances, according to an embodiment. The system 300 may include a body 302 having one or more chambers 341-347 therein. Although seven chambers 341-347 are shown, it will be appreciated that more or fewer chambers may be present. Each chamber 341-347 may include a fragrance accord 351-357 therein. The fragrance accords 351-357 may include those described above. For example, the fragrance accord 351 in chamber 341 may be fruit, and the fragrance accord 352 in chamber 342 may be green.

The system 300 may be configured to receive one or more signals from a portable electronic device 370. The portable electronic device 370 may be a cellular phone, a tablet, an MP3 player, another audio signal producing device, or the like. As shown, the body 302 may include an auxiliary input 304 configured to physically connect with a corresponding auxiliary output 372 of the portable electronic device 370. In other embodiments, the body 302 may include a receiver configured to receive wireless signals from the portable electronic device 370.

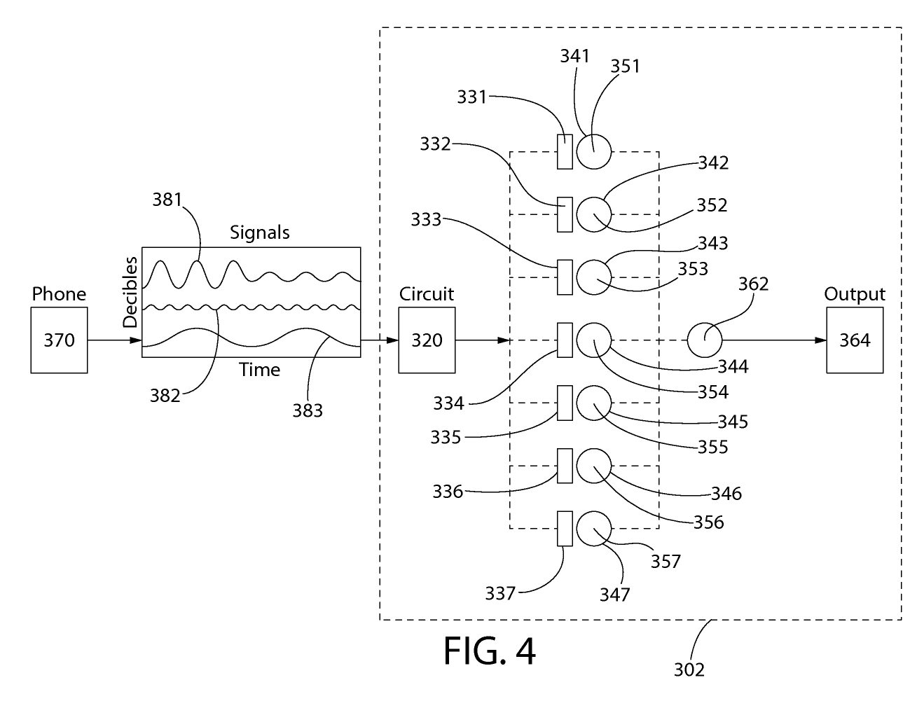

FIG. 4 depicts a schematic view of the system 300 shown in FIG. 3, according to an embodiment. Although not shown in FIG. 3, the body 302 may also include one or more circuits (one is shown 320). The circuit 320 may include components similar to those described above with respect to the circuit 120. The circuit 320 may be connected to one or more emitters (seven are shown: 331-337) in the body 302. Each of the emitters 331-337 may be positioned adjacent to and/or connected to a corresponding one of the chambers 341-347. The emitters 331-337 may be or include piezoelectric drivers, heating elements, aerosol devices, and the like.

Referring to FIGS. 3 and 4, in operation, the user may select an input (e.g., input 311 corresponding to the cucumber citrus fragrance) using the portable electronic device 370. For example, the user may download an application (i.e., an “app”) on the portable electronic device 370 that includes a list of one or more fragrances, or the user may navigate to a website including the list of fragrances. The user may then select the input 311 from the list by touching an icon on the screen of the portable electronic device 370 that identifies the fragrance, typing a command into the portable electronic device 370 that identifies the fragrance, or speaking a voice command into the portable electronic device 370 that identifies the fragrance. In another embodiment, the user may customize his or her own fragrance including two or more of the fragrance accords 351-357 rather than selecting from a predetermined list of fragrances.

In response to the selected input 311, the portable electronic device 370 may transmit one or more signals (three are shown: 381-383) to the circuit 320 in the system 300. The signals 381-383 may be transmitted through the auxiliary input 304 and output 372, or the signals 381-383 may be transmitted wirelessly. In at least one embodiment, the signals 381-383 may be audio signals (e.g., a song, tone, or combination of tones) corresponding to the selected input 311. The circuit 320 may receive and process the signals 381-383, and the circuit 320 may then transmit one or more signals that correspond to the signals 381-383 to one or more of the emitters 331-337. For example, the cucumber citrus fragrance may be made up from fragrance accords 351-353 but not fragrance accords 354-357. In this instance, the processing of the signals 381-383 may cause the circuit 320 may transmit signals to the emitters 331-333 causing the emitters 331-333 to activate (e.g., bend, vibrate, or otherwise displace). When the emitters 331-333 activate, they may cause or allow the corresponding chambers 341-343 to dispense a portion of the fragrance accords 351-353 contained therein.

In at least one embodiment, the cucumber citrus fragrance may be made up of equal portions of the fragrance accords 351-353. In this embodiment, the signals transmitted to the emitters 331-333 may cause the emitters 331-333 to vibrate or displace with the same amplitude, frequency, and/or duration. In another embodiment, the cucumber citrus fragrance may be made up of different portions of the fragrance accords 351-353. For example, the cucumber citrus fragrance may be made up of 40% of fragrance accord 351, 40% of fragrance accord 352, and 20% of fragrance accord 353. In this embodiment, the signals transmitted to the emitters 331, 332 may cause the emitters 331, 332 to vibrate or displace with twice the amplitude, frequency, and/or duration as the emitter 333 to output twice as much liquid as is output by the emitter 333.

The fragrance accords 351-353 dispensed from the chambers 341-343 may flow into a common chamber 362. The common chamber 362 may be a tubular passageway between the chambers 341-347 and an output 364. In at least one embodiment, the fragrance accords 351-353 may be stirred or mixed in the common chamber 362 prior to being dispensed through the output 364. The output 364 may be or include a nozzle configured to spray the fragrance into the air for the user to smell.

What is claimed is:

1. A system for producing a fragrance, comprising:
a body having a plurality of chambers defined therein, wherein each chamber includes a fragrance accord therein;
a circuit positioned within the body; and
a plurality of emitters positioned within the body, wherein the emitters are connected to and controlled by the circuit, and wherein each of the emitters is positioned adjacent to a corresponding one of the chambers and controls ejection of the fragrance accord therein.

2. The system of claim 1, wherein the emitters are selected from the group consisting of piezoelectric drivers, heating elements, and aerosol devices.

3. The system of claim 1, wherein the circuit is configured to receive a plurality of fragrance selections, each corresponding to a different fragrance.

4. The system of claim 3, wherein each fragrance comprises a different combination of two or more of the fragrance accords.

5. The system of claim 1, wherein, in response to receiving a fragrance selection, the circuit transmits a first signal to a first of the emitters, causing the first of the emitters to displace.

6. The system of claim 5, wherein, in response to the first of the emitters displacing, a portion of a first of the fragrance accords is dispensed from a first of the chambers adjacent to the first of the emitters.

7. The system of claim 6, wherein, in response to receiving the fragrance selection, the circuit transmits a second signal to a second of the emitters, causing the second of the emitters to displace.

8. The system of claim 7, wherein, in response to the second of the emitters displacing, a portion of a second of the fragrance accords is dispensed from a second of the chambers adjacent to the second of the emitters.

9. The system of claim 8, wherein the selected fragrance comprises the first fragrance accord and the second fragrance accord.

10. The system of claim Jany of the preceding claims, wherein the fragrance accords are in liquid form, gel form, or solid state form.

11. A system for producing a fragrance, comprising:
a body having a plurality of chambers defined therein, wherein each chamber includes a fragrance accord therein;
a circuit positioned within the body, wherein the circuit is configured to receive a signal corresponding to a fragrance selection from a portable electronic device; and
a plurality of piezoelectric drivers positioned within the body, wherein the piezoelectric drivers are connected to and controlled by the circuit, and wherein each of the piezoelectric drivers is positioned adjacent to a corresponding one of the chambers.

12. The system of claim 11, wherein the circuit is configured to receive the signal when the portable electronic device is physically connected to the body.

13. The system of claim 11, wherein the circuit is configured to receive the signal wirelessly from the portable electronic device.

14. The system of claim 11, wherein the signal comprises an audio signal.

15. The system of claim 11, wherein the signal comprises at least a portion of a song.

16. A method for producing a fragrance, comprising:
receiving a selection of a fragrance;
transmitting, in response to the selection, a first signal to a first piezoelectric device that causes the first piezoelectric device to displace, wherein, in response to the first piezoelectric device displacing, a first fragrance accord is dispensed from a first chamber; and
transmitting, in response to the selection, a second signal to a second piezoelectric device that causes the second piezoelectric device to displace, wherein, in response to the second piezoelectric device displacing, a second fragrance accord is dispensed from a second chamber, and wherein the fragrance comprises the first fragrance accord and the second fragrance accord.

17. The method of claim 16, where the selection is received from a portable electronic device.

18. The method of claim 17, wherein the selection is received wirelessly from the portable electronic device.

19. The method of claim 16, further comprising combining the first fragrance accord and the second fragrance accord in a common chamber to form the fragrance.

20. The method of claim 16, wherein the portable electronic device is selected from the group consisting of a cellular phone, a tablet, and an MP3 player.
